(19) United States
(12) Patent Application Publication
(10) Pub. No.: US 2013/0050639 A1
(43) Pub. Date: Feb. 28, 2013

(54) OBLIQUE-INCIDENCE DEPOSITED SILICON OXIDE LAYERS FOR DYNAMIC OPHTHALMIC LENSES

(71) Applicants: Anita Trajkovska, Christiansburg, VA (US); Joshua N. Haddock, Roanoke, VA (US); Ronald Blum, Roanoke, VA (US); Amitava Gupta, Roanoke, VA (US); Marko Theodoor Blom, NK Enschede (NL); Peter Tijssen, GW Enschede (NL)

(72) Inventors: Anita Trajkovska, Christiansburg, VA (US); Joshua N. Haddock, Roanoke, VA (US); Ronald Blum, Roanoke, VA (US); Amitava Gupta, Roanoke, VA (US); Marko Theodoor Blom, NK Enschede (NL); Peter Tijssen, GW Enschede (NL)

(73) Assignee: PixelOptics, Inc., Roanoke, VA (US)

(21) Appl. No.: 13/590,042

(22) Filed: Aug. 20, 2012

Related U.S. Application Data
(60) Provisional application No. 61/526,008, filed on Aug. 22, 2011.
(60) Provisional application No. 61/563,937, filed on Nov. 28, 2011.
(60) Provisional application No. 61/579,217, filed on Dec. 22, 2011.

(51) Int. Cl. G02C 7/02; A61F 2/16; B05D 5/06

(52) U.S. Cl. 351/159.39; 427/108; 427/109; 623/6.22

(57) ABSTRACT

An ophthalmic lens including an electro-active optical element including a substrate; a liquid crystalline material; and at least one first layer. The at least one first layer can include a layer of silicon oxide (SiOx) disposed between the liquid crystalline material and the substrate, and deposited onto a surface of the substrate at an oblique angle in reference to a plane normal to the mean surface of the substrate facing the liquid crystalline material.

### CROSS-REFERENCE TO RELATED APPLICATIONS

The present application claims priority to and incorporates by the reference in the entirety each of the following provisional patent applications: U.S. Prov. Pat App. No. 61/526,008, filed Aug. 22, 2011; U.S. Prov. Pat. App. No. 61/563,937, filed Nov. 28, 2011; and U.S. Prov. Pat. App. No. 61/579,217, filed Dec. 22, 2011.

### BACKGROUND

Adjustable focus eyeglasses are prescription eyeglasses with an adjustable focal length. They may compensate for refractive errors (such as presbyopia) by providing variable focusing, allowing users to adjust them for desired distance or prescription, or both. Current bifocals and progressive lenses are static, in that the user has to change their eye position to look through the portion of the lens with the focal power corresponding to the distance of the object. This usually means looking through the top of the lens for distant objects and down through the bottom of the lens for near objects. Adjustable focus eyeglasses have one focal length, but it is variable without having to change where one is looking. There are currently two basic methods to achieve variable focal length: electro-active and opto-mechanical. Electro-active lenses generally provide a region of adjustable optical power by changing the refractive index of an electro-active material (e.g., a liquid crystal material) by the application and removal of electrical power.

### SUMMARY

The technology includes ophthalmic lenses (such as an intra-ocular lens) including an electro-active optical element comprising a substrate, a liquid crystalline (LC) material, and at least one first layer. The first layer can be a layer of silicon oxide (SiOx) disposed between the LC material and the substrate, and deposited onto a surface of the substrate at an oblique angle in reference to a plane normal to the mean surface of the substrate facing the LC material. In some embodiments, the first layer comprises SiO, while in others, the first layer comprises SiO2. In some embodiments, the first layer has a thickness in the range of approximately 10 nm-200 nm.

In some embodiments, the first layer is a barrier layer, while in others the first layer is an electro-insulating layer. In some embodiments the first layer is an alignment layer, a barrier layer, and an insulating layer. In some embodiments, the oblique angle is an angle in the range substantially from 10 degrees to 80 degrees. In some embodiments, the substrate comprises a surface relief feature, e.g., one of: a diffractive feature, and refractive feature. In some embodiments, the lens does not comprise either of: a rubber polymer film or a photo-aligned film. In some embodiments, the first layer is one of: a sputtered layer, and an evaporated layer.

In some embodiments, the electro-active optical element further includes at least one second layer of silicon oxide, disposed between the first layer and the substrate, and deposited at a normal angle in reference to a plane parallel to the mean surface of the substrate facing the liquid crystalline material. In some embodiments, the second layer is a barrier layer. In some embodiments, the first layer and the second layer have a combined thickness in the range 10 nm-300 nm.

The technology includes methods for manufacturing an ophthalmic lens (such an in intra-ocular lens) including an electro-active optical element. In some embodiments the method includes depositing a first layer comprising a silicon oxide at an oblique angle in reference to a plane perpendicular to the mean surface of the substrate. In some embodiments, the substrate includes at least one of a surface relief diffractive element, and a surface relief refractive element. In some embodiments, the first layer is deposited by one of: evaporation and sputtering. In some embodiments, the first layer is deposited at an angle in the range substantially from 10° to 80°.

### DESCRIPTION OF THE DRAWINGS

FIG. 1 illustrates portions of an electro-active semi-finished lens blank (EASFLB).

FIG. 2 illustrates an exploded cross-sectional view of the EASFLB depicted in FIG. 1.

FIG. 3 illustrates an uncoated surface relief diffractive element on a substrate (FIG. 3A) and a section of a surface relief diffractive element on a substrate coated with an oblique SiOx layer (FIG. 3B) in accordance with embodiments of the present technology.

FIG. 4 illustrates the oblique “needle-like” layer topography of a SiOx layer on a substrate in accordance with embodiments of the present technology.

FIG. 5 illustrates a cross-sectional view of liquid crystal cell comprising nematic LC doped with a dichroic dye.

FIG. 6 and FIG. 7 illustrate a quantitative assessment of LC orientation in LC cells prepared with substrates having oblique-incidence deposited SiOx alignment layers.

FIG. 8 illustrates the composition of obliquely-evaporated SiOₓ layer, where x=2, in accordance with embodiments of the present technology.

### DETAILED DESCRIPTION

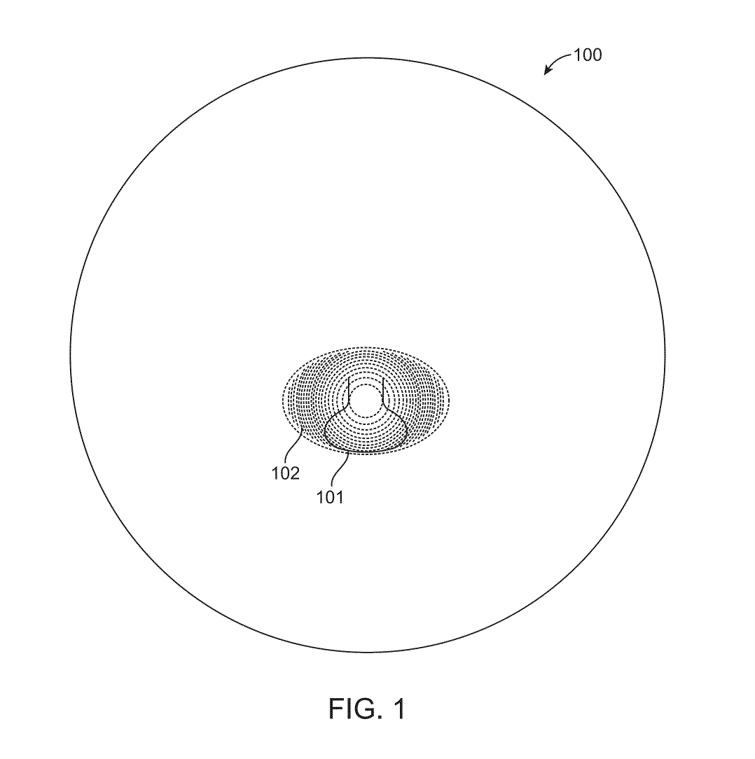

The technology disclosed herein finds use in dynamic ophthalmic lenses comprising an electro-active element, including by way of example only, contact lenses, spectacle lenses, and intra-ocular lenses. FIG. 1 illustrates an electro-active semi-finished lens blank (EASFLB) 100. The EASFLB 100 can comprise a first substrate (e.g., a top substrate) and a second substrate (e.g., a bottom substrate). FIG. 1 depicts a top view of the EASFLB 100. As depicted in FIG. 1, the EASFLB 100 can comprise a progressive addition optical power region 101 in optical communication with a dynamic, electro-active, diffractive optical power region 102. The dynamic, electro-active, diffractive optical power region 102 can comprises an electro-active material such as, for example, a cholesteric liquid crystalline (CLC) material. The electro-active material can be encapsulated within a volume by the two bounding substrates (i.e., the top and bottom substrates of the EASFLB 100).

The dynamic, electro-active, diffractive optical power region 102 is shown as having an oval shape but is not so limited. The dynamic, electro-active, diffractive optical power region 102 can be of any shape (e.g., round, flat-topped, semi-circle, etc.) and can be blended as described in U.S. patent application Ser. No. 12/166,526, filed Jul. 2, 2008, which is hereby incorporated by reference in its entirety. An adhesive can adhere the two substrates of the EASFLB 100 together and can be applied via one or more fill ports (not shown). Electrical contacts (not shown) can allow a voltage to be applied to the dynamic, electro-active, diffractive optical power region 102 so as to allow activation of the dynamic, electro-active, diffractive optical power region 102. Electrical contact can be made between the electrical contacts and the dynamic, electro-active, diffractive optical power region 102 via transparent conductors. The electrical contacts can be applied to the inner surfaces of the two bounding substrates and can therefore be embedded within the EASFLB 100.

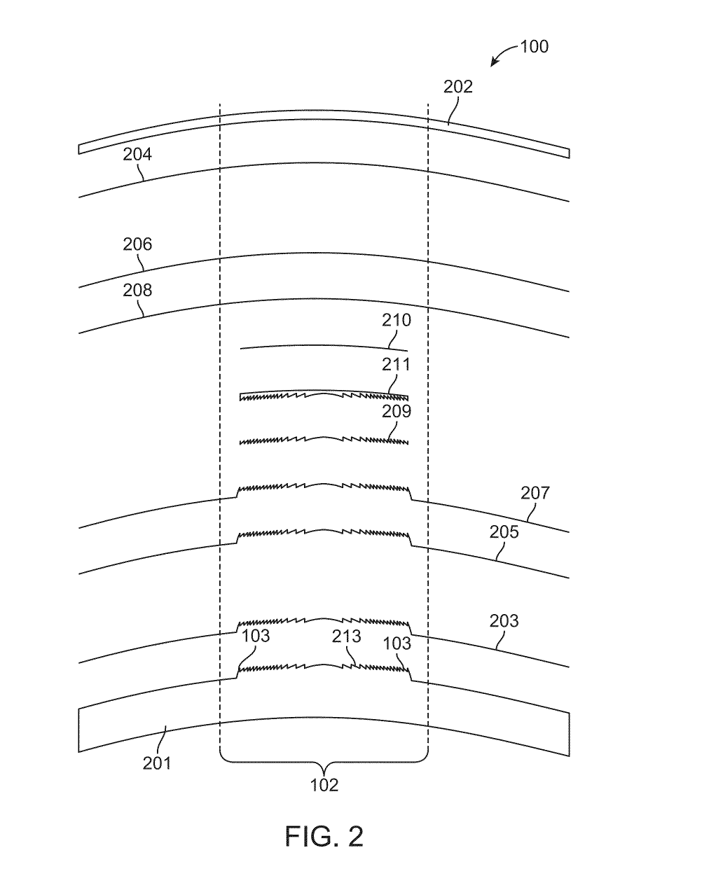

An exploded cross-sectional view of the EASFLB 100 (not to scale) is shown in FIG. 2. The EASFLB 100 can be constructed from the aforementioned bounding substrates—in particular, a back substrate 201 and a front substrate 202. The back substrate 201 can be thicker than the front substrate 202. The back substrate 201 can comprise any lens material. As an example, the back substrate 201 can comprise a material having a refractive index of 1.67 such as Mitsui MR-10. The front substrate 202 can also comprise any lens material. As an example, the front substrate 202 can comprise the same lens material as the back substrate 201, e.g., the front substrate 202 can comprise MR-10 material. Alternatively, the front substrate 202 can comprise a different lens material, e.g., the back substrate 201 can comprise Trivex® having a refractive index of 1.53. As will be appreciated by one skilled in the relevant arts, the features and characteristics of the front substrate 202 and the back substrate 201 can be interchanged.

The anterior, convex surface of the back substrate 201 can contain a surface relief diffractive structure 213. The surface relief diffractive structure 213 can be implemented as a multi-order surface relief diffractive structure as described in U.S. patent application Ser. No. 12/118,226, filed on May 9, 2008, which is hereby incorporated by reference in its entirety. The posterior, concave surface of the back substrate 201 can be substantially featureless.

The anterior, convex surface of the front substrate 202 can comprise the progressive optical power region 101 and semi-visible fiducial marks (not shown) while the concave surface of the front substrate 202 can be substantially featureless. The front substrate 202 can also comprise the adhesive fill ports (not shown). Alternatively or in addition thereto, the back substrate 201 can comprise the adhesive fill ports.

Additional layers and structures can be applied to the convex surface of the back substrate 201 and to the concave surface of the front substrate 202 to allow operation of the dynamic, electro-active, diffractive optical power region 102. First layers 203 and 204 can any transparent material that is electrically insulating. As an example, the layers 203 and 204 can comprise SiOx (e.g., Si02 or Si03). Each of the layers 203 and 204 can have a thickness of 20 nm for example.

On top of each of the layers 203 and 204, a conductive material can be patterned into fine wires to form the electrical contacts. On top of the electrical contacts, transparent conductor layers 205 and 206 can be deposited. Each of the transparent conductor layers 205 and 206 can comprise a transparent conductive material such as Indium Tin Oxide (ITO) or Zinc Oxide (ZnO). The transparent conductor layers 205 and 206 can have a thickness of 20 nm for example. The transparent conductor layers 205 and 206 can be in electrical contact with the corresponding electrical contacts. The electrical contacts can provide electrical contact to the dynamic, electro-active, diffractive optical power region 102 through the edge of the EASFLB 100.

One or more of the transparent conductor layers can be deposited or formed to be patterned electrode structures (or pixelated structures) as described in U.S. patent application Ser. No. 12/246,543, filed on Oct. 7, 2008 and U.S. patent application Ser. No. 12/135,587, filed on Jun. 9, 2008, both of which are hereby incorporated by reference in their entirety. Such a patterned electrode structure can be used to form a desired diffractive pattern using a volume of electro-active material (e.g., electro-active material 211 contained in a space that need not rest on top of a diffractive relief structure).

On top of the transparent conductor layers 205 and 206, insulating layers 207 and 208 can be deposited. The insulating layers 207 and 208 can comprise any transparent material that is electrically insulating. As an example, the layers 207 and 208 can comprise SiOx (e.g., similar to the first layers 203 and 204). The insulating layers 207 and 208 can comprise 170 nm of SiOx for example. The final layers deposited can comprise liquid crystal alignment material layers 209 and 210 which act to align a volume of electro-active material 211 encapsulated within the EASFLB 100. The arrangement and thicknesses of the layers 203-210 can increase the luminous transmittance through the EASFLB 100 while decreasing electrical power consumption of the dynamic, electro-active, diffractive optical power region 102.

The surface relief diffractive structure 213 and the layers and elements 203-211 can be considered to be part of an electro-active element of the EASFLB 100 (e.g., the dynamic, electro-active, diffractive optical power region 102). Any of the layers and elements 203-211 can be deposited across an entire area of the EASFLB 100 (e.g., the insulating layers 203 and 204) or can be deposited over less than an entire area of the EASFLB 100 or a portion of the entire area of the EASFLB 100 (e.g., the alignment layers 209 and 210). Further, the surface relief diffractive structure 213 can occupy any portion of the anterior, convex surface of the back substrate 201. Additionally, as will be appreciated by one skilled in the relevant arts, the surface relief diffractive structure (and associated electro-active material seal feature and adhesive seal feature for example) of the EASFLB 100 can be alternatively positioned on the front substrate 202.

As shown in FIG. 2, the dynamic, electro-active, diffractive optical power region 102 is shown as comprising multiple layers and elements of the EASFLB 100. Further, the dynamic, electro-active, diffractive optical power region 102 is shown as occupying a portion of an entire horizontal width of the EASFLB 100. As described further below, the EASFLB 100 can be further processed to form a finished lens blank or an edged lens (ready to be mounted into a spectacle frame). Overall, the arrangement of the layers of the EASFLB 100 can be varied as will be understood by one skilled in the relevant arts and as described in U.S. patent application Ser. No. 12/042,643, filed on Mar. 3, 2008, which is hereby incorporated by reference in its entirety.

The liquid crystal (LC) material 211 can undergo change in its optical characteristics under an applied electrical field. For successful operation and performance of LC-based devices, the LC material 211 should be appropriately aligned, e.g., using alignment layers 209 and 210, both in the presence of an electric field, and in the absence of an electric field. Changes in the optical characteristics of the LC material 211 occur exclusively due to different orientations of LC molecules in different states of device operation (e.g. ON-state, OFF-state, and many states in-between). Depending on the operation mode of LC device, one can distinguish the so-called “field-free orientation” (OFF-state) and a range of LC orientations under the applied voltage (ON-state).

The field-free LC material 211 orientation is substantially determined by the boundary conditions of a geometry confining the LC material 211, which boundary conditions can be dictated by the alignment layers 209, 210. The basis for the molecular orientation is the physical and/or chemical anisotropy on the surface of alignment layers 209, 210 resulting in an anisotropic arrangement of the adjacent LC molecules in the LC layer 211.

Conventionally, LC alignment is created by the unidirectional mechanical rubbing of polymer films with a rubbing cloth. This method has been widely used due to its simplicity, durability, and low-cost. However, the generation of dust and electrostatic surface charge during the rubbing, as well as mechanical surface defects, can be detrimental for device performance and lifetime. The debris generation is not in line with the clean-room requirements, while the high processing temperature of polyimide alignment films limits their application on many plastic substrates. Further, organic polymers may lose their alignment when heated at or above their glass transition temperature T_{g}, typically in the range of 30° C.-100° C. for many organic polymers. This factor becomes even more important when considering implantable electro-active lenses. Also, the rubbing alignment process introduces variation in the level of alignment and is hard to control precisely, especially on surface relief features.

Another method is the alignment on SiOₓ layers deposited at oblique incident angles on substantially flat surfaces. SiOx layers are can provide thermal and photochemical stability. Depending on the deposition conditions, variety of LC orientations can be achieved: from no-pre-tilt in-plane LC alignment to high-pre-tilt and vertical LC alignment. SiOx deposition can be done at ambient temperature, or slightly elevated temperatures above the ambient temperature.

Among alternatives, the most promising is photo-alignment. Photo-alignment uses polarized light to generate chemical anisotropy on photo-reactive surfaces via directional photo-reaction (e.g., isomerization, anisotropic cross-linking, or directional photo-degradation). Anisotropic inter-molecular interaction between different surface molecular species has been shown to be sufficient to align LC molecules. Photo-alignment offers the possibility of micro-patterning via photo-mask for multi-domain LC orientations, as well as feasibility on flexible substrates. However, the majority of photo-alignment materials suffer from long-term stability, viz. light-, thermal- and chemical instability, making them non-suitable for many applications.

With regard to electro-active ophthalmic lenses that can be implanted in a recipient, such lenses are required to be sterilized prior to implantation. The sterilization process may cause the implant to heat up to and beyond 45° C. or higher. Consequently, the use of an inorganic alignment layer is preferred for application in implants. A preferred method to fabricate an inorganic alignment layer is by an oblique deposition of SiOx, resulting in a needle-like surface morphology.

In embodiments of the present technology, an obliquely-deposited SiOₓ layer can perform one or more roles on a surface-relief optical feature: as a barrier layer, and as an LC alignment layer. In such embodiments, electro-active optical elements, such as dynamic ophthalmic lens, light shutter and so on, can use obliquely-deposited SiOₓ layers on surface relief diffractive optics or refractive optics.

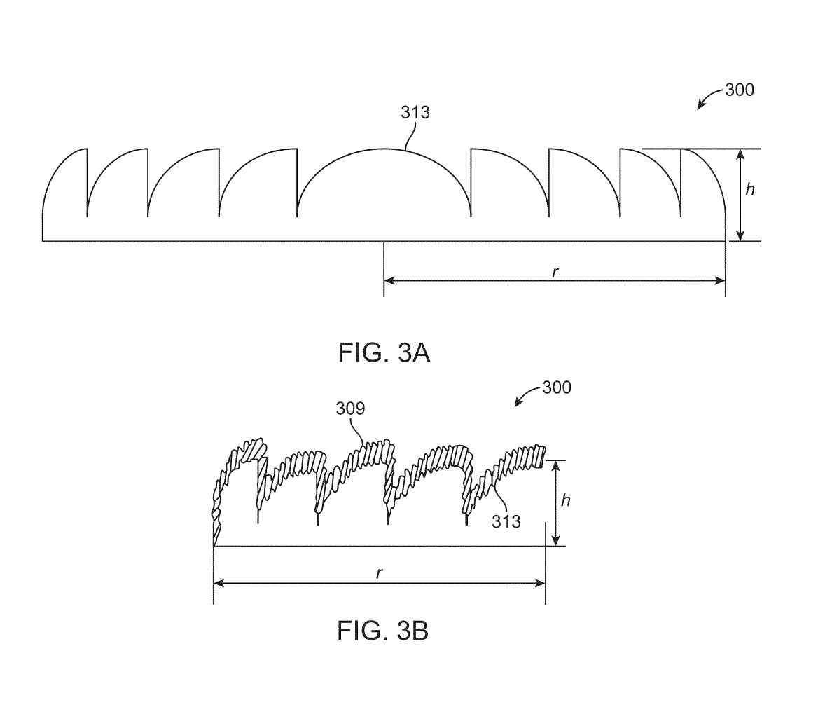

Oblique SiOₓ layers can be deposited via oblique sputtering or oblique evaporation. Oblique SiOₓ layers can be applied solely in thickness range from approximately 5 nm-200 nm, or in a combination with normally-deposited SiOₓ layers (normal incidence to the mean plane of the surface deposited on) in total thickness range from 10 nm-300 nm. FIG. 3A is a schematic representation 300 of a surface relief diffractive element 313 similar to surface relief feature 213. FIG. 3B is a schematic representation of an obliquely-deposited SiOₓ layer 309 deposited on the surface relief diffractive element 313.

When depositing an SiOₓ layer acting only as an insulating/barrier layer in electro-active optical lens, it is deposited at normal incidence in the range between 20-200 nm. In those applications, in order to provide the necessary LC molecular orientation in OFF-state, electro-active lenses utilize a rubbed or photo-aligned polymer layer on top of SiO₂ layer. As stated above, both alignment methods, rubbing method and photo-alignment, suffer from major disadvantages. Obliquely-deposited SiOₓ layers can overcome these disadvantages by providing clean, debris-free, thermally- and photo-chemically-stable alignment layers.

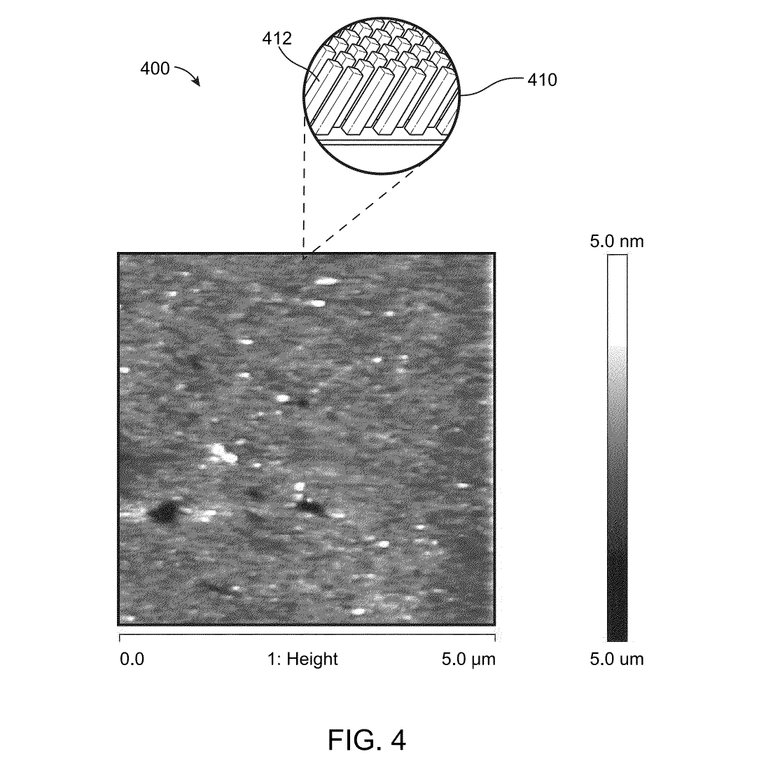

In embodiments of the present technology, the SiOₓ morphology, surface topography and roughness, as well as the chemical composition can be changed by varying: deposition angle (from approximately 10°-approximately 80°), deposition rate (1-10 Angstroms/s.), power (50-300 W) and working pressure. SiOₓ layers deposited at different conditions can lead to in-plane (no pre-tilt or low pre-tilt) LC alignment to vertical (90° or high pre-tilt) LC alignment. As shown in FIG. 4, the oblique “needle-like” layer topography 400, deposited at rate of 3 Angstroms/s., temperature of 30° C. and total chamber pressure of 2E-5 Ton yields certain pre-tilt in the orientation of overlaying LC layer. Detail 410, shows a simplified view of obliquely-deposited SiOx 412.

By introducing oxygen during the deposition from Si-target, the Si:O ratio can be changed, yielding variety of SiOₓ layer compositions (1≦x≦2). Different SiOₓ layer compositions will have different surface energies, which will affect the layer anchoring strength, and thus, the orientation of overlaying LC molecules.

Use of obliquely-deposited SiOₓ as an alignment layer can provide an alignment layer that is stable to 250° C. and higher, allowing the electro-active lens 102 to be hermetically sealed in the EASFLB 100 at relatively high temperature. Also, the fabrication of the alignment layer can be automated and integrated with the deposition of a transparent electrode layer and a resistive layer, e.g., a resistive layer of SiOₓ. Such an approach can provide anchoring energy similar to that of polyamides.

In an exemplary embodiment, a substrate, such as substrate 201, was fabricated from mineral glass (Ohara: refractive index 1.64) and one wall of the substrate was etched to form a diffractive optic consisting of a phase-wrapped Fresnel lens. The surface containing the diffractive optic was coated with a layer of resistive material, then over-coated with a transparent electrode material Indium Tin Oxide (ITO). A second wall also was coated with SiOx and ITO. These substrates were then vapor deposited with SiOx as follows.

SET 1—Vapor deposition was performed at oblique angles, where the deposition angle is defined with respect to the surface normal. The deposition angles were 20°, 30°, and 40°. The ITO layer thickness was 20 nm, and the oblique-deposited SIOx thickness was 10 nm. The substrate was Ohara high index glass in 100 mm rounds, 0.3 mm thick. This was diced to 50 mm square pieces, which were coated with ITO without any masking. Before the subsequent SiOx runs, a shadow mask was added in order to provide electrical contact to the ITO. The deposition equipment consisted of a SiOx evaporation machine with 4 substrate positions (50 mm square, now converted to 100 mm square), in which the deposition angle is adjustable.

SET 2—Vapor deposition was performed in which the deposition angle is defined with respect to the normal on the surface. The deposition angles were 20°, 30°, and 40°. The ITO layer thickness was 20 nm, a standard deposition SiOx layer (in the direction of the substrate normal) was 90 nm, and the oblique-deposited SIOx thickness was 10 nm. The substrate was Ohara high index glass in 100 mm rounds, 0.3 mm thick. This was diced to 50 mm square pieces, which were coated with ITO without any masking. Before the subsequent SiOx runs, a shadow mask was added in order to provide electrical contact to the ITO. The deposition equipment consisted of a SiOx evaporation machine with 4 substrate positions (50 mm square, now converted to 100 mm square), in which the deposition angle is adjustable.

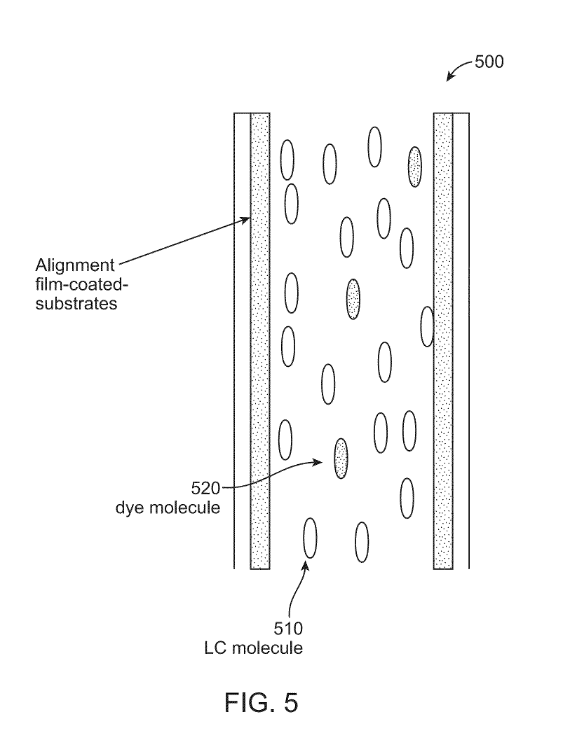

Referring to FIG. 5, a cross-sectional view of LC 500 cell comprising nematic LC 510 doped with a dichroic dye 520 is illustrated schematically. The cells of Set 1 were filled and doped in such manner. The dichroic dye 520 was used to track the orientation of the neighboring LC molecules 510 (under the assumption that the dye molecules 520 orient along with the LC molecules 510).

LC alignment can be quantitatively expressed in terms of orientation order parameter S=(R−1)/(R+2); where R is the ratio between Aₚₐᵣₐ and Aₚₑᵣₚ, i.e., Aₚₐᵣₐ/Aₚₑᵣₚ, where Aₚₐᵣₐ is the polarized dye absorption at the absorption maximum parallel to the alignment direction, and Aₚₑᵣₚ is the polarized dye absorption at the absorption maximum perpendicular to the alignment direction. The orientational order parameter can have values from S=−0.5 (when LC molecules orient perpendicular to the alignment direction) to S=1.0 (when all LC molecules orient perfectly parallel to the alignment direction). Table 1 presents the result of the study across sample Set 1 compared to samples prepared using photo-alignment.

TABLE 1

| Alignment | R = Aₚₐᵣₐ/Aₚₑᵣₚ | S = (R − 1)/(R + 2) |
|---|---|---|
| Photo-alignment | R = 6.58 | S = 0.64 |
| Oblique SiOx | R = 3.78 | S = 0.48 |
| 20 deg | R = 6.00 | S = 0.63 |
| Oblique SiOx | R = 4.42 | S = 0.53 |
| 30 deg | R = 4.16 | S = 0.51 |
| Oblique SiOx | R = 2.77 | S = 0.37 |
| 40 deg | R = 3.80 | S = 0.48 |

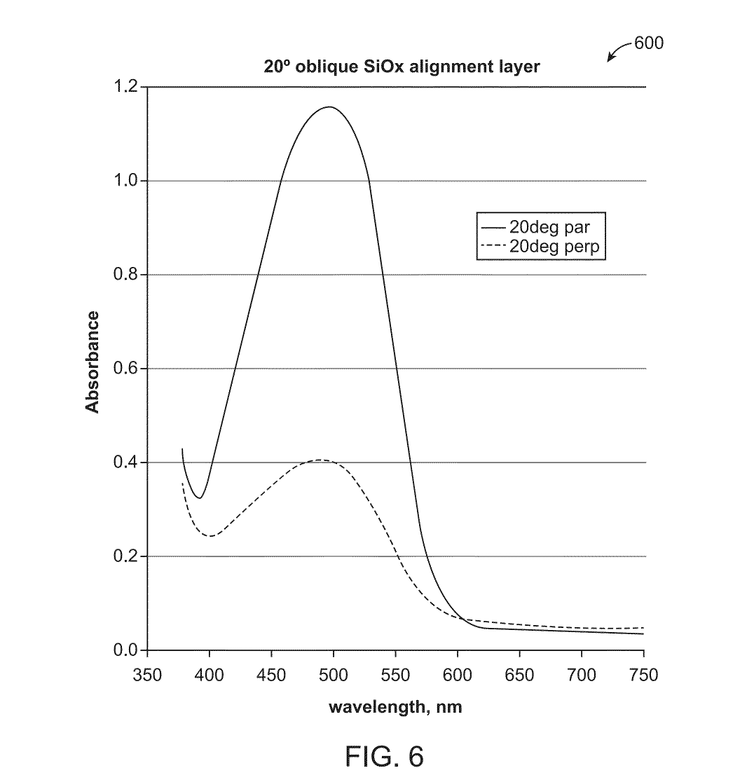

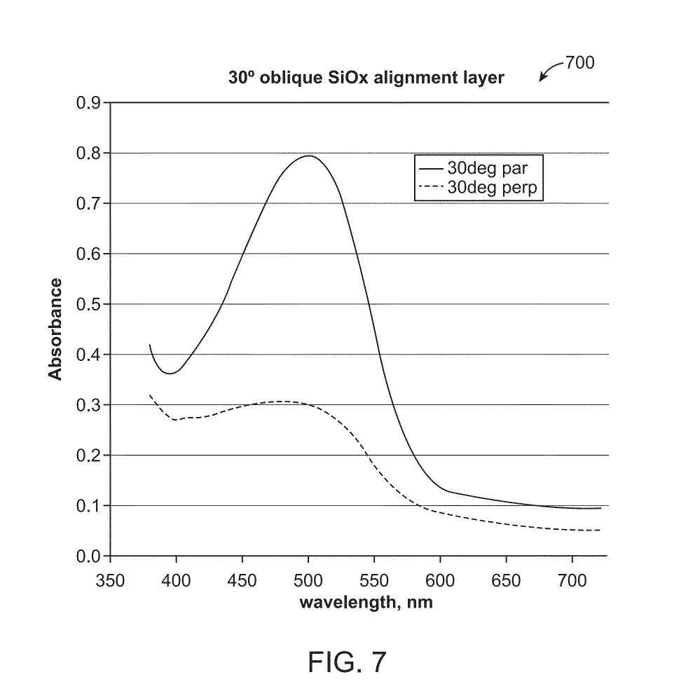

LC orientation also can be assessed directly by the polarized ultra-violet/visible (UV-VIS) absorption of the dye 520 in the LC/dye mixture parallel and then perpendicular to the alignment direction. FIG. 6 and FIG. 7 illustrate a quantitative assessment 600, 700 of LC orientation in Set 1 LC cells prepared with substrates having oblique-incidence deposited SiOx alignment layers. The cells were filled with nematic LC (MDA-98-160) doped with Disperse red 1 dye. Absorbance was measured across a range of linearly polarized incident light wavelengths from 350 nm to 750 nm for LC oriented at 20° parallel and 20° perpendicular to the director, FIG. 6; and for 30° parallel (field free orientation) and 30° perpendicular (ON state) FIG. 7.

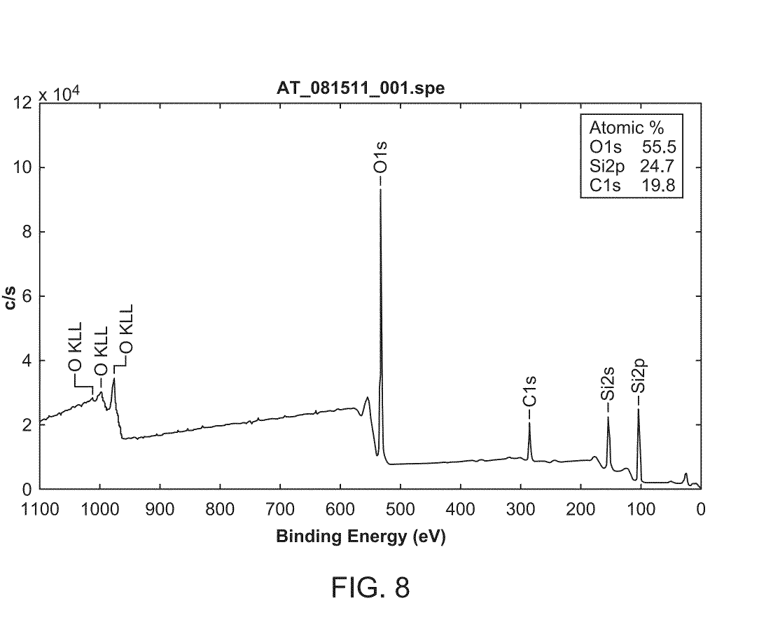

The surface characteristics of the exemplary oblique SiOx alignment layers were examined using x-ray photo-electron spectroscopy (XPS) and atomic force microscopy. Surface chemical composition of the exemplary layers was assessed by XPS. Two types of XPS scans were performed: a survey/elemental XPS scan, and a high-resolution Si-band XPS scan. The survey XPS scan revealed the elements present on the surface of the SiOx layers (Si, C, and O), From the peak ratio O/Si one can calculate the x-value in SiOx. For all SiOx layers, deposited at 20°, 30°, and 40°, x was found to be 2, i.e., the composition of the investigated layers is SiO₂. High resolution Si-band XPS scans can also reveal the source of the Si, i.e., if Si originates from SiO₂ or from another oxide (SiOx, where x<2). From the values it can be concluded that the Si originates form SiO₂. FIG. 8 shows the composition of obliquely-evaporated SiOₓ layer, where x=2.

The surface topography of the exemplary obliquely-deposited SiOx alignment layers was analyzed by AFM. The surface topography for each sample was similar. In general, it appears that higher deposition angles give smoother surfaces.

In some embodiments, the present technology finds application in implantable electro-active devices. For example, electro-active intra-ocular lenses incorporating a surface relief diffractive optic in optical communication with a liquid crystal based dynamic index matching medium can benefit from a liquid crystal alignment layer not processed by physical rubbing. Optically processed alignment (i.e., photo-alignment) layers are an attractive option but due to their low glass transition temperature (T_{g}) will not tolerate the high temperature processes associated with sealing and sterilization of an implantable medical device (e.g., laser welding and autoclaving, respectively). Obliquely-deposited SiOx alignment layers are a better solution, as such layers can sustain higher temperature due to the fact that they are inorganic glasses.

In some embodiments of the present technology an oblique SiOₓ layer can be deposited by evaporation or sputtering at oblique angles ranging from 10°-80°, and used solely as an insulating/barrier layer in electro-active diffractive or refractive optical element, or in a combination with SiOₓ layer deposited at normal incidence. In some embodiments, an oblique-incidence-evaporated SiOₓ layer can be deposited on a surface relief diffractive substrate or refractive substrate to be used as a liquid crystal alignment layer in electro-active surface relief optical element. In some embodiments, an oblique-incidence SiOₓ layer can be sputtered on a surface relief diffractive substrate or refractive substrate to as a liquid crystal alignment layer in electro-active surface relief optical element.

In some embodiments, an obliquely-deposited SiOₓ layer can have a double role as an insulating/barrier layer and as a liquid crystal alignment layer in dynamic diffractive or refractive optical element, such as optical lens, ophthalmic lens, light shutter, light filter, etc. In some such embodiments, an electro-active optical element with obliquely-deposited SiOₓ layer is made with a liquid crystal layer and without rubbed polymer film or photo-aligned film.

In some embodiments of the present technology, an electro-active cell can include an obliquely-deposited silicon oxide layer for use as a dynamic and switchable optic in an intraocular implant. In some such embodiments, the obliquely-deposited SiOx layer can be deposited directly on a transparent electrode. In some such embodiments, the obliquely-deposited SiOx layer can be deposited on a SiOx substrate deposited by conventional means. In some such embodiments, the cell can be made of mineral glass. In mineral glass embodiments, the electro-active cell can be sealed by using a high temperature glass sealing process subsequent to deposition of the obliquely-deposited SiOx layer; and the cell can be subjected to a temperature higher than 150° C. In some embodiments, the obliquely-deposited SiOx can be deposited using chemical vapor deposition.

In some embodiments, an intra-ocular implant comprises an electro-active cell including an obliquely-deposited silicon oxide layer. In some such embodiments, the obliquely-deposited silicon oxide layer is deposited directly on a transparent electrode. In some such embodiments, the obliquely-deposited silicon oxide layer is deposited on a silicon oxide layer deposited by conventional means. In some such embodiments, the cell includes substrates made of mineral glass. In some of those embodiments, the electro-active cell is sealed by using a high temperature (e.g., >150° C.) glass sealing process subsequent to deposition of the obliquely-deposited silicon oxide layer.

In some embodiments, the obliquely-deposited silicon oxide layer is deposited at an angle of more than 0° and less than 45°. In some embodiments the obliquely-deposited silicon oxide layer is deposited using a chemical vapor deposition process. In some embodiments, the obliquely-deposited silicon oxide layer is deposited in a thickness in excess of approximately 5 nm, but less than approximately 200 nm. In some such embodiments, the obliquely-deposited silicon oxide layer is deposited to a thickness of approximately 10 nm.

While various embodiments of the present technology have been described above, it should be understood that they have been presented by way of example and not limitation.

These applications can be that of, by way of example only, by way of example only, electronic focusing eyeglasses, electro-active eyeglasses, fluid lenses being activated by way of an electronic actuator, mechanical or membrane lenses being activated by way of electronics, electro-chromic lenses, electronic fast tint changing liquid crystal lenses, lenses whose tint can be altered electronically, lenses that by way of an electrical charge can resist or reduce the attraction of dust particles, lenses or eyeglass frames housing or having an electronic display affixed thereto, electronic eyewear providing virtual reality, electronic eyewear providing 3-D capabilities, electronic eyewear providing gaming, and electronic eyewear providing augmented reality.

Overall, it will be apparent to one skilled in the pertinent art that various changes in form and detail can be made therein without departing from the spirit and scope of the technology. Therefore, the present technology should only be defined in accordance with the following claims and their equivalents.

1. An ophthalmic lens comprising:
an electro-active optical element comprising:
a substrate;
a liquid crystalline material; and
at least one first layer:
comprising a layer of silicon oxide (SiOx),
disposed between the liquid crystalline material and the substrate, and
deposited onto a surface of the substrate at an oblique angle in reference to a plane normal to the mean surface of the substrate facing the liquid crystalline material.

2. The ophthalmic lens of claim 1, wherein the first layer comprises SiO.

3. The ophthalmic lens of claim 1, wherein the first layer comprises SiO₂.

4. The ophthalmic lens of claim 1, wherein the first layer has a thickness in the range 10 nm-200 nm.

5. The ophthalmic lens of claim 1, wherein the first layer is a barrier layer.

6. The ophthalmic lens of claim 1, wherein the first layer is an electro-insulating layer.

7. The ophthalmic lens of claim 1, wherein the first layer is an alignment layer, a barrier layer, and an insulating layer.

8. The ophthalmic lens of claim 1, wherein the oblique angle is an angle in the range substantially from 10 degrees to 80 degrees.

9. The ophthalmic lens of claim 1, wherein the substrate comprises a surface relief feature.

10. The ophthalmic lens of claim 9, wherein the surface relief feature comprises one of: a diffractive feature, and refractive feature.

11. The ophthalmic lens of claim 1, wherein the lens does not comprise either of: a rubber polymer film or a photo-aligned film.

12. The ophthalmic lens of claim 1, wherein the first layer is one of: a sputtered layer, and an evaporated layer.

13. The ophthalmic lens of claim 1, wherein the electro-active optical element further comprises:
at least one second layer:
comprising a layer of silicon oxide,
disposed between the first layer and the substrate, and
deposited at a normal angle in reference to a plane parallel to the mean surface of the substrate facing the liquid crystalline material.

14. The ophthalmic lens of claim 13, wherein the second layer is a barrier layer.

15. The ophthalmic lens of claim 13, wherein the first layer and the second layer have a combined thickness in the range 10 nm-300 nm.

16. A method for manufacturing an ophthalmic lens, the method comprising: depositing a first layer comprising a silicon oxide at an oblique angle in reference to a plane perpendicular to the mean surface of a substrate of an electro-active optical element of the ophthalmic lens.

17. The method of claim 16, wherein the substrate comprises one of: a surface relief diffractive element, and a surface relief refractive element.

18. The method of claim 16, wherein the first layer is deposited by one of: evaporation and sputtering.

19. The method of claim 16, wherein the first layer is deposited at an angle in the range substantially from 10° to 80°.

20-34. (canceled)

35. The ophthalmic lens of claim 1, wherein the ophthalmic lens is an intra-ocular lens.
